(19) United States
(12) Patent Application Publication
(10) Pub. No.: US 2019/0375787 A1
(43) Pub. Date: Dec. 12, 2019

(54) PEPTIDOMIMETICS AND THEIR USE IN THERAPY

(71) Applicant: INSTYTUT HOMEOSTAZY SP. Z O.O., Warszawa (PL)

(72) Inventors: Andrzej Wojciech LIPKOWSKI, Warszawa (PL); Aleksandra MISICKA-KESIK, Pruszków (PL); Piotr SOSNOWSKI, Czerwin (PL); Anna Katarzyna PUSZKO, Warszawa (PL); Anna LASKOWSKA, Pruszków (PL); Marek DURLIK, Warszawa (PL); Krzysztof RÓZYCKI, Warszawa (PL)

(21) Appl. No.: 16/464,532

(22) PCT Filed: Nov. 28, 2017
(86) PCT No.: PCT/PL2017/050057

§ 371 Date: May 28, 2019

(30) Foreign Application Priority Data
Nov. 28, 2016 (PL) P.419611
Nov. 28, 2016 (PL) P.419612

(51) Int. Cl. C07K 5/107; C07K 5/087

(52) U.S. Cl. CPC C07K 5/1016; A61K 45/06; C07K 5/0812

(57) ABSTRACT

Peptidomimetic analogs suitable for use in cancer therapy, or as antagonists of opioid drugs, and suitable application of the compounds are disclosed. The described compounds display cytostatic and cytotoxic effects toward intestinal cancer cells, and pancreatic cancer cells, display affinity to μ and δ receptors, and act as antagonists of opioid drugs. Particularly, they are intended for administration to gastrointestinal system, in the form of tablets, infusions, injections, or implants, in therapy and supporting therapy of intestinal or pancreatic cancer, and in elimination of respiratory depression triggered by opioids, and to assure appropriate intestine peristalsis during an opioid therapy.

### TECHNICAL FIELD

The invention relates to analogs of peptidomimetics suitable for use in therapy of intestinal cancer or pancreatic cancer, as antagonists of opioid drugs, and to use of the compounds in therapy of intestinal cancer or pancreatic cancer. The described compounds demonstrate cytostatic and cytotoxic activity against cancer cells, especially intestinal cancer or pancreatic cancer, demonstrate affinity toward μ and δ receptors and act as antagonists of opioid drugs. The compounds are intended to be administrated by various ways, especially by gastrointestinal tract, in the form of tablets, infusions, injections, or implants, in treatment and supporting therapy of cancer, especially intestinal cancer or pancreatic cancer, and in ensuring normal intestinal peristalsis during opioid therapy.

### Background of the Invention

Fluoropyrimidines, mainly 5-fluorouracil (5-FU), are commonly applied in treatment of intestinal cancer, as well as other cancers. The drug is often administered in combination with leucovorin as bolus injection or as intravenous infusion (Humeniuk R., et. al., 2009). 5-FU is often used in chemotherapy in combination with other drugs, such as oxaliplatin, which suppress DNA replication and induce cellular apoptosis (Raymond E., Faivre S., et. al., 1998; Raymond E., Chaney S. G., et. al., 1998), or irinotecan, working as topoisomerase I inhibitor (Akhtar R., et. al., 2014). Capecitabin constitutes another popular treatment in intestinal cancer therapy, it is administered orally and absorbed by mucous membrane of the gastrointestinal tract (Pentheroudakis G., et. al., 2002).

Pancreatic ductal adenocarinoma; (PDAC) is a malignant cancer having a very poor prognosis, even if diagnosed at early stages. Nowadays, PDAC is the seventh most common cause of death from cancer, and it is prognosticated that in the coming years the number of PDAC cases will be growing (Conroy T, Bachet JB, Ayav A et al. European Journal of Cancer 2016; 57:10-22). Current chemotherapy of PDAC includes gemcitabine, abraxane (nab-paclitaxel), 5-fluorouracil, erlotinib, or combination therapy FOLFIRINOX. Nab-paclitaxel in combination with gemcitabine have become a new standard in treatment of metastatic pancreatic cancer (inoperable). Chemotherapy bears a big risk of side effects, such as hair loss, nausea, vomit or weakness. Currently, combination therapy is often used, and various chemotherapies, or a chemotherapy and different methods of therapy are used together.

A part of the patients is treated by chemotherapy following a surgery, to eliminate cancer cells not removed by the surgery (adjuvant therapy). In cases with very poor prognosis (inoperable pancreatic cancer) chemotherapy is used as a palliative care, focused at improving of life comfort of patients.

Gemcitabine is a nucleoside analog, which due to the similarity to 2′-deoxycitidine is built into DNA instead of it. In result, the synthesis of DNA strand is disrupted and the cell dies. Gemcitabine constitutes the most often recommended drug in treatment of pancreatic cancer.

Abraxan is an albumin bound paclitaxel. Paclitaxel displays antimitotic activity by inhibition of microtubule depolimerization, in result separation of sister chromatids during the cell division is blocked. The dysfunction of mitosis results in cell death.

5-Fluorouracil is a fluorinated derivative of pyrimidine. The 5-fluorouracil is transformed in cell into biologically active metabolites: phospho-deoxyribonucleotide (5-dUMP) and fluorouridine triphosphate (FUTP). 5-dUMP blocks the thymidylate synthetase, and in result production of thymidylic acid, a component of DNA, is also blocked. FUTP is incorporated into RNA and blocks uracil phosphatase, what results in RNA with an incorrect structure. The dysfunction of DNA and RNA synthesis leads to a damage and death of the cell.

Erlotinib acts as tyrosine kinase inhibitor of receptor type I of human epidermal growth factor (EGFR or HER1). It inhibits phosphorylation of EGFR inside the cell, what leads to inhibition of cell division and/or the cell death.

The therapy FOLIFIRINOX depends on simultaneous treatment with four chemotherapeutics—folinic acid (FOL), 5-fluorouracil (F), irinotecan (IRIN) and oxaliplatin (OX).

Unfortunately, gemcitabine and abraxan demonstrate poor efficiency and onerous side effects, characteristic also for FOLIFIRINOX, which is a mixture of potentially very toxic compounds (Conroy T, Desseigne F, Ychou M et al., The New England Journal of Medicine 2011; 364:1817-1825). In addition, in recent years a growing number of gemcitabine resistance cases is observed (Xie J, Jia Y, Genes & Diseases 2015; 2(4):299-306).

For the above reasons, providing of new compounds suitable for treating and/or prophylaxis of pancreatic cancer is highly needed. Unexpectedly, the aim was achieved in the present invention.

Very often, cancer therapy is accompanied by administration of analgesic drugs, applied at all stages of tumor development, and as the disease progresses their potency must be increased. In especially difficult cases, when surgery or pharmacological treatment is no longer effective or is impossible, in the terminal period, the therapy is limited to relieve of pain.

For years, opioids have been considered as the most potent analgesic agents, and morphine is one of the most often prescribed.

Strong painkilling activity of morphine have caused, that it has been commonly used in therapy of all kinds of cancer. For this reason, it has been of interest how morphine influence the cancer itself. Until now, the results have been unclear. It was demonstrated, inter alia, that clinically efficient doses of morphine stimulated grow of breast cancer in mice model (Bimonte S., et. al., 2015; Nguyen J., et. al., 2014); this was connected with acceleration of angiogenesis, inhibition of apoptosis, and progression of cell cycle (Farooqui M., et. al., 2007). Similar conclusion was drawn from experiments using mice models of sarcoma and leukemia (Ishikawa M., et. al., 1993). Moreover, it was found both in in vitro and in vivo studies, that tumor growth might be supported by occasional application of large doses of morphine, or its prolonged application in small doses (Zong J., et. al., 2000). What's more, morphine can inhibit apoptosis of cancer cell (Lin X., et. al., 2007) or induce tumor growth by increase in expression of cyclooxygenase-2 (COX-2) (Farooqui M., et. al., 2006; Salvemini D., et. al., 1993; Arerangaiah R., et. al., 2007; Nédélec E., et. al., 2001), and/or by stimulation of prostaglandin E2 mediated angiogenesis (Chang S. H., et. al., 2004; Griffin R. J., et. al., 2002; Salvemini D., et. al., 1994; Leahy K. M., et. al., 2002).

The invention is aimed at providing access to new compounds suitable for treatment and prophylaxis of cancer, especially intestinal cancer or pancreatic cancer, with simultaneous elimination of adverse interactions of opioids with opioid receptors in gastrointestinal tract, and restoring/maintaining intestine motility during a morphine therapy.

The present invention is particularly aimed at elimination of adverse effects associated with simultaneous treatment of patients with anticancer drugs and morphine as a painkiller, especially in therapy of intestinal cancer. In case of this diseases, about 75% of the patients require continuous anti-pain therapy. In addition, treatment of a chronic pain is associated with fast development of tolerance to drug, and in a consequence with a need for increased doses of analgesics, that in turn, might intensify the negative side effects (Hiliger M. Wspóczesna onkologia 2001; 5(4):168-174). Some of the opioid anelgesics, e.g. morphine, demonstrate also cancer stimulating activity (Farooqui M, Li Y, Rogers T et al., British Journal of Cancer 2007; 97(11):1523-1531), and in a consequence decrease effectiveness of the used anticancer therapy.

### The Essence of Invention

The present invention relates to compounds of general formula:

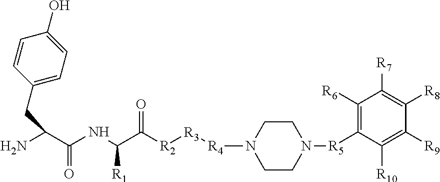

wherein:
R₁ relates to side chain of D-amino acid, selected from: D-Ala, D-Tre, D-Ser, D-Met, D-Leu, D-Glu, D-Asp, D-Lys or D-Arg,
R₂ relates to none or to L-amino acid residue selected from Gly or Lys, or dipeptide residue L-Gly-L-Lys,
R₃ relates to L-amino acid residue selected from Phe or Trp,
R₄ relates to none or L-amino acid residue Lys,
R₅ relates to straight, saturated, or unsaturated hydrocarbon chain of general formula CₙHₘ, wherein n is an integer from 1 to 4, and m is an even integer from 2 to 10,
R₆₋₁₀ relates independently to a substituent selected from: hydrogen, hydroxyl, methyl, formyl, carboxy, methoxycarbonyl, carbamoyl, cyano, amino, methylamino, dimethylamino, iodo, fluoro, nitro, or sulphone,
for use in therapy or prophylaxis of cancer, especially intestinal or pancreatic cancer.

Preferably, R₂ relates to L-Gly residue, when:
a) R₄ relates to L-Lys residue, or
b) R₅ does not relate to a hydrocarbon chain of formula C₂H₂, or
c) at least one from R₆-R₁₀ does not relate to hydrogen.

Preferably, a compound according to the present invention is selected from a group consisting of:

tyrosyl-D-alanyl-glicyl-cinnamylpiperazine,
tyrosyl-D-threonyl-glicyl-phenylalanyl-cinnamylpiperazine,
tyrosyl-D-arginyl-cinnamylpiperazine, and tyrosyl-D-threonyl-cinnamylpiperazine,
L-tyrosyl-D-alanyl-L-phenylalanyl-cinnamylpiperazine,
L-tyrosyl-D-alanyl-L-tryptophyl-cinnamylpiperazine,
L-tyrosyl-D-threonyl-L-phenylalanyl-cinnamylpiperazine,
L-tyrosyl-D-threonyl-L-tryptophyl-cinnamylpiperazine,
L-tyrosyl-D-alanyl-glicyl-L-phenylalanyl-cinnamylpiperazine,
L-tyrosyl-D-alanyl-glicyl-L-tryptophyl-cinnamylpiperazine,
L-tyrosyl-D-threonyl-glicyl-L-phenylalnyl-cinnamylpiperazine,
L-tyrosyl-D-threonyl-glicyl-L-tryptophfyl-cinnamylpiperazine.

Preferably, compound to be used according to the invention, is used in a form intended for oral, by injection, or intravenous administration. Preferably, it is used in a form of oral tablet to be administered directly into gastrointestinal tract, as intravenous infusion for peripheral administration, or injection into the tumor and its vicinity.

Preferably, compound to be used according to the invention, is used in a form of multidrug pharmaceutical composition, especially containing additionally an analgesic opioid, and/or anticancer drug.

Preferably, compound to be used according to the invention, is intended for use in classical chemotherapy of intestinal cancer, applied prior to surgery (neo-adjuvant therapy), or post-surgery (adjuvant therapy), or in chemotherapy of inoperable intestinal cancer, or as a supporting therapy, or is intended to be used in therapy of other cancers, to protect the intestine from metastasis of cancer of different tissue origin.

Preferably, compound to be used according to the invention, is intended for use in classical chemotherapy of pancreatic cancer, applied prior to surgery (neo-adjuvant therapy), or post-surgery (adjuvant therapy).

Another embodiment of the invention relates to compound of general formula:

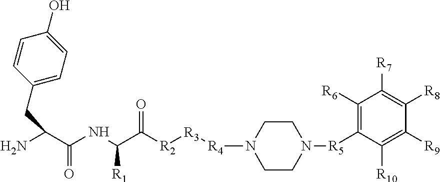

wherein:
R₁ relates to side chain of D-amino acid, selected from: D-Ala, D-Tre, D-Ser, D-Met, D-Leu, D-Glu, D-Asp, D-Lys or D-Arg,
R₂ relates to none or to L-amino acid residue selected from Gly or Lys, or dipeptide residue L-Gly-L-Lys,
R₃ relates to L-amino acid residue selected from Phe or Trp,
R₄ relates to none or L-amino acid residue Lys,
R₅ relates to straight, saturated or unsaturated hydrocarbon chain of general formula CₙHₘ, wherein n is an integer from 1 to 4, and m is an even integer from 2 to 10,
R₆₋₁₀ relates independently to a substituent selected from: hydrogen, hydroxyl, methyl, formyl, carboxy, methoxycarbonyl, carbamoyl, cyano, amino, methylamino, dimethylamino, iodo, fluoro, nitro, or sulphone,
except of compound which is already known from the Polish patent application no P.402324, of general formula:

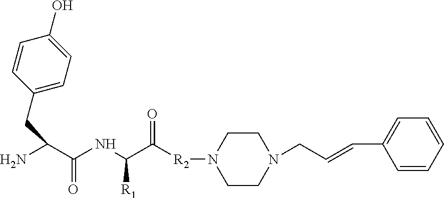

R₁ relates to side chain of D-amino acid selected from: D-alanine, D-threonine, D-serine, D-methionine, D-leucine, D-glutamine, D-asparagine, D-lysine, or D-arginine,
and R₂ relates to none, or residue of glycine, or a dipeptide selected from Gly-Phe or Gly-Trp.

In a further embodiment, the invention relates to use of the described above, new compound, as an antagonist of opioid drugs, particularly to eliminate the opioid drugs side effects, such as intestinal disorder or constipation, and to assure appropriate intestine peristalsis, through opioid antagonistic activity. Compounds according to the invention can also be used to eliminate a risk of respiratory depression, associated with the use of opioids.

Preferably, the compound is intended for oral or peripheral application, to eliminate constipation due to opioid drugs. Equally preferably, the compound is intended for direct or indirect interaction with opioid receptors, especially peripheral receptors, located outside the central nervous system. Equally preferably, the compound is used in the form of oral tablet for direct administration to gastrointestinal system, or in the form of intravenous infusion for peripheral administration. Equally preferably, the compound is used in the form of multidrug pharmaceutical composition, preferably containing an analgesic opioid. Equally preferably, the compound is used in a form of composition containing a polymeric carrier of the active agent.

Unexpectedly, it was found that compounds according to the invention, except antagonistic activity toward opioids, exhibited also cytotoxic and cytostatic activity toward intestinal and pancreatic cancer cells. Thereby, in a particular embodiment, the invention combines the effect of protection of intestine from adverse influence of opioids on intestine motility, with ability to cure or prevent intestinal and pancreatic cancer, and ability to prevent development of intestinal cancer in result of metastasis.

### DETAILED DESCRIPTION OF INVENTION

In general, the invention relates to peptidomimetics suitable for application in treatment and profilaxy of intestinal cancer and pancreatic cancer, and in assuring normal motility of intestine during opioid therapy, while some of the compounds are known from the Polish patent application no P.402324.

In relation to definitions of R, used in the present description, the side chain of D-amino acid is understood as the group attached to a carbon of the amino acid, in case of D-Ala this is understood as the group indicated in the formula below:

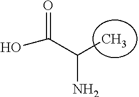

as the side chain of D-Thr is understood:

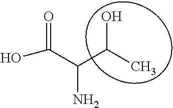

as the side chain of D-Ser is understood:

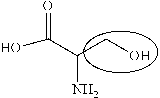

as the side chain of D-Met is understood:

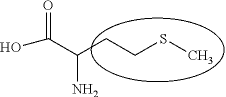

as the side chain of D-Leu is understood:

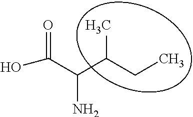

as the side chain of D-Gln is understood:

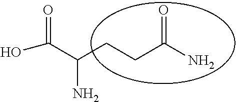

as the side chain of D-Asn is understood:

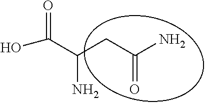

as the side chain of D-Lys is understood:

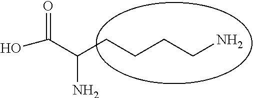

as the side chain of D-Arg is understood:

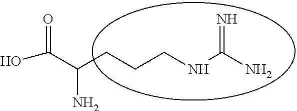

Whereas, in relation to definitions of groups R₂ and R₄ used in the description, when the group is defined as none, it means that the right side atom (nitrogen or carbon) connected to the group is bonded directly to the left side carbon atom connected with the group. With reference to the remaining definitions of R₂, in the case where it relates to:

L-Gly, it should be understood as:

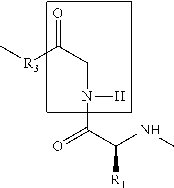

L-Lys, it should be understood as:

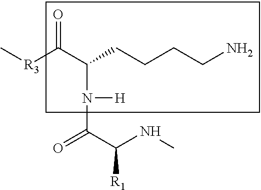

L-Gly-L-Lys, it should be understood as:

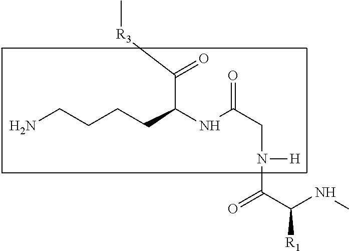

With reference to the definition of R₃, in the case where it relates to:

L-Phe, it should be understood as:

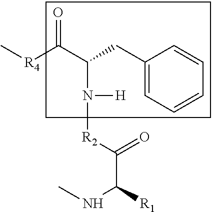

L-Trp, it should be understood as:

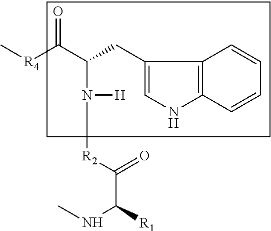

With reference to the definition of R₄, in the case where it relates to:

L-Lys, it should be understood as:

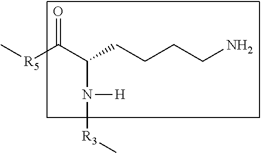

In search for new peptidomimetics of opioid, which should display a high affinity for opioid receptor, particularly opioid receptor μ, and at the same time act as antagonists for opioid drugs, such as morphine, fentanyl, or opioid peptides, such as encephalin, or bifalina, it was unexpectedly found that the obtained peptide analogs of opioids display also cytostatic activity toward intestinal cancer cells and pancreatic cancer cells. The activity was manifested both through cytostatic effect and cell death, as well as through loosening of tumor structure, which lead to easier penetration of active agents into the tumor.

The examples given below illustrate the cytostatic and cytotoxic effects according to the invention, toward intestinal or pancreatic cancer cells. Examples of interaction with opioid receptors are also given.

Peptidomimetics according to the invention can be obtained using methods known in the art, particularly using the method described in the Polish patent application no P.402324. The given below examples of synthesis of exemplary peptidomimetics, may be easily adopted by a person skilled in the art, to obtain any compound according to the invention,

a) Tyrosyl-D-alanyl-glicyl-cinnamylpiperazine hydrochloride

Trans-1-cinnamylpiperazine was acylated with t-Boc-Tyr-D-Ala-Gly using N,N-dicyclohexylcabodiimide
N-hydroxysuccinimide coupling method in N,N-dimethylformamide solution. N,N-Dicyclohexylurea was filtered off, and the crude intermediate was precipitated with water. The solid was washed three times with water and dried. t-Butyloxycarbonyl protecting group was removed using 5% hydrochloride in ethyl acetate. The crude end-product was precipitated as the hydrochloride with ethyl ether, and purified by preparative HPLC in a gradient of 0.5% hydrochloric acid/etanol. Pure tyrosyl-D-alanyl-glicyl-cinnamylpiperazine hydrochloride was isolated as the product.
   b) Tyrosyl-D-threonyl-glicyl-phenylalanyl-cinnamylpiperazine hydrochloride

Trans-1-cinnamylpiperazine was acylated with t-Boc-Tyr-D-Thr-Gly-Phe, using N,N-dicyclohexylcabodiimide—N-hydroxysuccinimide coupling method in N,N-dimethylformamide solution. N,N-Dicyclohexylurea was filtered off, and the crude intermediate was precipitated with water. The solid was washed three times with water and dried, t-Butyloxycarbonyl protecting group was removed using 5% hydrochloride in ethyl acetate. The crude end-product was precipitated as the hydrochloride with ethyl ether, and purified by preparative HPLC in a gradient of 0.5% hydrochloric acid/etanol. Pure tyrosyl-D-threonyl-glicyl-phenylalanyl-cinnamylpiperazine hydrochloride was isolated as the product.

c) Tyrosyl-D-arginyl-cinnamylpiperazine dihydrochloride

Trans-1-cinnamylpiperazine was acylated with t-Boc-Tyr-D-Arg, using N,N-dicyclohexylcabodiimide-N-hydroxysuccinimide coupling method in N,N-dimethylformamide solution. N,N-Dicyclohexylurea was filtered off, and the crude intermediate was precipitated with water. The solid was washed three times with water and dried. t-Butyloxycarbonyl protecting group was removed using 5% hydrochloride in ethyl acetate. The crude end-product was precipitated as the hydrochloride with ethyl ether, and purified by preparative HPLC in a gradient of 0.5% hydrochloric acid/etanol. Pure tyrosyl-D-arginyl-cinnamylpiperazine dihydrochloride was isolated as the product.

Particularly preferred compounds according to the invention selected from:

tyrosyl-D-alanyl-glicyl-cinnamylpiperazine,
tyrosyl-D-threonyl-glicyl-phenylalanyl-cinnamylpiperazine,
tyrosyl-D-arginyl-cinnamylpiperazine, and tyrosyl-D-threonyl-cinnamylpiperazine,
L-tyrosyl-D-alanyl-L-phenylalanyl-cinnamylpiperazine,
L-tyrosyl-D-alanyl-L-tryptophyl-cinnamylpiperazine,
L-tyrosyl-D-threonyl-L-phenylalanyl-cinnamylpiperazine,
L-tyrosyl-D-threonyl-L-tryptophyl-cinnamylpiperazine,
L-tyrosyl-D-alanyl-glicyl-L-phenylalanyl-cinnamylpiperazine,
L-tyrosyl-D-alanyl-glicyl-L-tryptophyl-cinnamylpiperazine,
L-tyrosyl-D-threonyl-glicyl-L-phenylalanyl-cinnamylpiperazine,
L-tyrosyl-D-threonyl-glicyl-L-tryptophyl-cinnamylpiperazine,
can be prepared analogously.

The listed above compounds are prepared by initial synthesis of Boc-L-Tyr-D-Ala, or Boc-L-Tyr-D-Thr, or Boc-L-Tyr-D-Ala-Gly, or Boc-L-Tyr-D-Thr-Gly, and Boc-Tyr-D-Arg on 2-chlorotrityl resin, using Fmoc-protected amino acids, and HATU/DIPEA methodology for the coupling reactions, and 20% piperidine in DMF for deprotection. The product are cleaved from the solid support with AcO:TFE:DCM mixture, and used for acylation of phenylalanyl-trans-cinnamylpiperazine or tryptophyl-trans-cinnamylpiperazine, by TBTU/DIPEA method. The t-butyloxycarbonyl protecting group is removed using TFA:DCM mixture.

To better illustrate cytostatic and cytotoxic activity of the compounds according to the present invention toward cancer cells, especially intestinal and pancreatic cancer cells, suitable examples are presented below. Examples illustrating interaction of the compounds with opioid receptors are also given. The interaction is important to assure normal intestine peristalsis.

### Example 1

Phenylalanyl-trans-1-cinnamylpiperazine was acylated with t-Boc-Tyr-D-Ala using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to Cl⁻ using the Dowex ion-exchange resin. The purified product, tyrosyl-D-alanyl-phenylalanyl-cinnamylpiperazine hydrochloride (further called TyrDAlaPheCyn) was tested on human pancreatic cancer cell line CFPAC-1, in comparison to commercial drugs, gemcitabine (gem) and 5-fluorouracil (5FU). The cells were grown on IMDM medium supplemented with 10% (v/v h.i. FBS, 2 mM L-glutamine and 1% (v/v) penicillin-streptomycin, in 37° C., in humidified atmosphere, with 5% CO₂. The cells were seeded in 96 wells plate (3×10³ cells per well) and incubated for 24 h (37° C., 5% CO₂) in the culture medium. Next, TyrDAlaPheCyn, gem and 5FU in 5% DMSO solutions were added, to give in wells concentrations 0 (5% DMSO); 0.01; 0.05; 0.1; 0.5; and 1 mM. The plates were incubated for 24 h (37° C., 5% CO₂). Cell viability was determined using colorimetric test MTS (3-(4,5-dimethylthiazol-2-yl)-5-(3-carboxymethoxyphenyl)-2-(4-sulfophenyl)-2H-tetrazolium). To each well, 20 μl of the reagent was added, and the plates were incubated in the same conditions for 1 h. The absorbance intensity at 490 nm was read. It was found that at concentrations 0.01-0.1 mM activities of the tested compound, gem and 5FU were similar. However, at higher concentrations, activity of the tested compound was even 4 times higher than for the reference drugs.

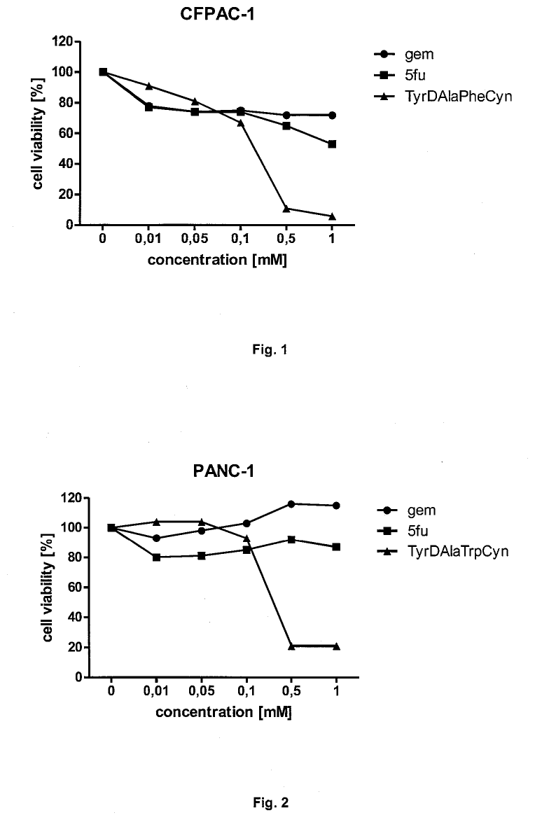

FIG. 1 presents a graph of human pancreatic cancer cells CFPAC-1 viability against concentrations of the used compounds, after 24 h of incubation.

### Example 2

Tryptophyl-trans-1-cinnamylpiperazine was acylated with t-Boc-Tyr-D-Ala using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to Cl⁻ using the Dowex ion-exchange resin. The purified product, tyrosyl-D-alanyl-tryptophyl-cinnamylpiperazine hydrochloride (further called TyrDAlaTrpCyn) was tested on human pancreatic cancer cell line CFPAC-1, in comparison to commercial drugs, gemcitabine (gem) and 5-fluorouracil (5FU). The cells were grown on DMDM medium supplemented with 10% (v/v h.i. FBS, 2 mM L-glutamine and 1% (v/v) penicillin-streptomycin, in 37° C., in humidified atmosphere, with 5% CO₂. The cells were seeded in 96 wells plate (3×10³ cells per well) and incubated for 24 h (37° C., 5% CO₂) in the culture medium. Next, TyrDAlaTrpCyn, gem and 5FU in 5% DMSO solutions were added, to assure in wells concentrations 0 (5% DMSO); 0.01; 0.05; 0.1; 0.5; and 1 mM. The plates were incubated for 24 h (37° C., 5% CO₂). Cell viability was determined using colorimetric test MTS (3-(4,5-dimethylthiazol-2-yl)-5-(3-carboxymethoxyphenyl)-2-(4-sulfophenyl)-2H-tetrazolium). To each well 20 μl of the reagent was added, and the plates were incubated in the same conditions for 1 h. The absorbance intensity at 490 nm was read. It was found that at concentrations 0.01-0.1 mM activities of the tested compound, gem and 5FU were similar. However, at higher concentrations, activity of the tested compound was even 4 times higher than for the reference drugs.

FIG. 2 presents a graph of human pancreatic cancer cells CFPAC-1 viability against concentrations of the used compounds, after 24 h of incubation.

### Example 3

Inhibition of cell migration was evaluated using 24-well plates with PET (polyethylene terephthalate) membrane transwell insert. The experiment was performed using IC₅₀ concentrations of the tested compounds, and migration of the cells, that means, the number of the cells passing through the membrane to the bottom side of the membrane, was observed. The cells which passed the membrane, were stained with crystal violet and counted.

### A) TyrDAlaGlyPheCyn

Phenylalanyl-trans-1-cinnamylpiperazine was acylated with t-Boc-Tyr-D-Ala-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to Cl⁻ using the Dowex ion-exchange resin. The purified product, tyrosyl-D-alanyl-glicyl-phenylalanyl-1-cinnamylpiperazine hydrochloride, was further called TyrDAlaGlyPheCyn. The compound was used for cellular tests:
For HT29 (intestinal cancer) cells, inhibition of cell migration was evaluated until 70%, for concentrations below 400 μM.
For Colo205 (intestinal cancer) cells, inhibition of cell migration was evaluated until 60%, for concentrations below 400 μM.
For SW480 (intestinal cancer) cells, inhibition of cell migration was evaluated until 60%, for concentrations below 400 μM.
For SW620 (intestinal cancer) cells, inhibition of cell migration was evaluated until 40%, for concentrations below 450 μM.

### B) TyrDAlaGlyTrpCyn

Tryptophyl-trans-1-cinnamylpiperazine was acylated with t-Boc-Tyr-D-Ala-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to Cl⁻ using the Dowex ion-exchange resin. The purified product, tyrosyl-D-alanyl-glicyl-tryptophyl-1-cinnamylpiperazine hydrochloride, was further called TyrDAlaGlyTrpCyn. The compound was used for cellular tests:
For HT29 (intestinal cancer) cells, inhibition of cell migration was evaluated until 50%, for concentrations below 400 μM.
For Colo205 (intestinal cancer) cells, inhibition of cell migration was evaluated until 40%, for concentrations below 400 μM.
For SW480 (intestinal cancer) cells, inhibition of cell migration was evaluated until 30%, for concentrations below 400 μM.
For SW620 (intestinal cancer) cells, inhibition of cell migration was evaluated until 30%, for concentrations below 500 μM.

### C) TyrDThrGlyPheCyn

Phenylalanyl-trans-1-cinnamylpiperazine was acylated with t-Boc-Tyr-D-Thr-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to CF using the Dowex ion-exchange resin. The purified product, tyrosyl-D-threonyl-glicyl-phenylalanyl-1-cinnamylpiperazine hydrochloride, was further called TyrDThrGlyPheCyn. The compound was used for cellular tests:
For SW480 (intestinal cancer) cells, inhibition of cell migration was evaluated until 45%, for concentrations below 500 μM.

### D) TyrDThrGlyTrpCyn

Tryptophyl-trans-1-cinnamylpiperazine was acylated with t-Boc-Tyr-D-Thr-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to Cl⁻ using the Dowex ion-exchange resin. The purified product, tyrosyl-D-threonyl-glicyl-tryptophyl-1-cinnamylpiperazine hydrochloride, was further called TyrDThrGlyTrpCyn. The compound was used for cellular tests:
For SW480 (intestinal cancer) cells, inhibition of cell migration was evaluated until 40%, for concentrations below 600 μM.
For SW620 (intestinal cancer) cells, inhibition of cell migration was evaluated until 30%, for concentrations below 600 μM.

### Example 4

Influence of the compounds on the cell growth was evaluated by colorimetric method, using MTT or XTT reagent, values of IC₅₀ were determined. The tests were based on reduction of the salt by mitochondrial dehydrogenase from metabolically active cells. The colored product of the reaction was determined colorimetrically, and in result the number of living cells was assigned.

A) TyrDAlaGlyPheCyn (the compound was prepared analogously as described in Example 1)
For HT29 (intestinal cancer) IC₅₀ was determined for concentrations below 400 μM.
For Colo205 (intestinal cancer) IC₅₀ was determined for concentrations below 400 μM.
For HCT116 (intestinal cancer) IC₅₀ was determined for concentrations below 400 μM.
For SW480 (intestinal cancer) IC₅₀ was determined for concentrations below 400 μM.
For SW620 (intestinal cancer) IC₅₀ was determined for concentrations below 450 μM.
   B) TyrDAlaGlyTrpCyn (the compound was prepared analogously as described in Example 1)
For HT29 (intestinal cancer) IC₅₀ was determined for concentrations below 400 μM.
For Colo205 (intestinal cancer) IC₅₀ was determined for concentrations below 400 μM.
For HCT116 (intestinal cancer) IC₅₀ was determined for concentrations below 400 μM.
For SW480 (intestinal cancer) IC₅₀ was determined for concentrations below 400 μM.
For SW620 (intestinal cancer) IC₅₀ was determined for concentrations below 500 μM.
   C) TyrDThrGlyPheCyn (the compound was prepared analogously as described in Example 1)
For HCT116 (intestinal cancer) IC₅₀ was determined for concentrations below 600 μM.
For SW480 (intestinal cancer) IC₅₀ was determined for concentrations below 600 μM.
For SW620 (intestinal cancer) IC₅₀ was determined for concentrations below 650 μM.
   D) TyrDThrGlyTrpCyn (the compound was prepared analogously as described in Example 1)
For HT29 (intestinal cancer) IC₅₀ was determined for concentrations below 600 μM.
For Colo205 (intestinal cancer) IC₅₀ was determined for concentrations below 650 μM.
For HCT116 (intestinal cancer) IC₅₀ was determined for concentrations below 600 μM.
For SW480 (intestinal cancer) IC₅₀ was determined for concentrations below 600 μM.
For SW620 (intestinal cancer) IC₅₀ was determined for concentrations below 600 μM.

### Example 5

Tryptophyl-trans-1-cinnamylpiperazine was acylated with t-Boc-Tyr-D-Ala-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to Cl⁻ using the Dowex ion-exchange resin. The purified product, tyrosyl-D-alanyl-glicyl-tryptophyl-1-cinnamylpiperazine hydrochloride, was further called TyrDAlaGlyTrpCyn.

Tryptophyl-trans-1-cinnamylpiperazine was acylated with t-Boc-Tyr-D-Thr-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to Cl⁻ using the Dowex ion-exchange resin. The purified product, tyrosyl-D-threonyl-glicyl-tryptophyl-1-cinnamylpiperazine hydrochloride, was further called TyrDThrGlyTrpCyn.

Phenylalanyl-trans-1-cinnamylpiperazine was acylated with t-Boc-Tyr-D-Ala-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to CFI using the Dowex ion-exchange resin. The purified product, tyrosyl-D-alanyl-glicyl-phenylalanyl-1-cinnamylpiperazine hydrochloride, was further called TyrDAlaGlyPheCyn.

Phenylalanyl-trans-1-cinnamylpiperazine was acylated with t-Boc-Tyr-D-Thr-Gly using TBTU/DIPEA methodology. The reaction product was precipitated with 10% NaHCO₃ and separated from the reaction mixture by filtration. The solid was washed three times with water, until neutral pH of the filtrate was reached. The t-butyloxycarbonyl group was removed using TFA:DCM (1:1). The crude product was purified by preparative HPLC in a gradient of water/methanol with addition of 0.1% trifluoroacetic acid. The counterion was changed to CFI using the Dowex ion-exchange resin. The purified product, tyrosyl-D-threonyl-glicyl-phenylalanyl-1-cinnamylpiperazine hydrochloride, was further called TyrDThrGlyPheCyn.

Affinity of the compounds for opioid receptors was evaluated by a radioisotope method, which depended on competitive binding of 0.5 nM selective radioactive agonists of the receptors, for μ: [3H]DAMGO and for δ: [3H]DELT, in the presence of increasing concentrations of the studied compounds in non-labeled forms (10-10, 5-10-6). The following values were found:

TyrDAlaGlyPheCyn—for the receptor μ below 100, and for the receptor δ below 300
TyrDAlaGlyTrpCyn—for the receptor μ below 100, and for the receptor δ below 300
TyrDThrGlyPheCyn—for the receptor μ below 100, and for the receptor δ below 300
TyrDThrGlyTrpCyn—for the receptor μ below 100, and for the receptor δ below 300.

The results indicated that the studied compounds were able to bond to the studied receptors.

1. Compound of general formula: 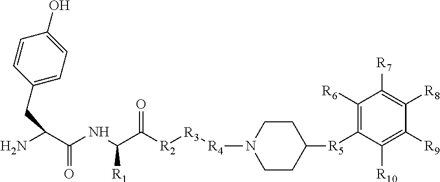
wherein:
R₁ relates to a side chain of D-amino acid selected from: D-Ala, D-Tre, D-Ser, D-Met, D-Leu, D-Glu, D-Asp, D-Lys or D-Arg,
R₂ relates to none, or to L-amino acid residue selected from Gly or Lys, or dipeptide residue L-Gly-L-Lys,
R₃ relates to L-amino acid residue selected from Phe or Trp,
R₄ relates to none or to L-amino acid residue Lys,
R₅ relates to straight, saturated or unsaturated hydrocarbon chain of general formula CₙHₘ, wherein n is an integer from 1 to 4, and m is an even integer from 2 to 10,
R₆₋₁₀ relates independently to a substituent selected from: hydrogen, hydroxyl, methyl, formyl, carboxy, methoxycarbonyl, carbamoyl, cyano, amino, methylamino, dimethylamino, iodo, fluoro, nitro, or sulphone,
for use in therapy or prophylaxis of cancer, especially intestinal or pancreatic cancer.

2. Compound for use according to claim 1, wherein, R₂ relates to L-Gly residue, when:
a) R₄ relates L-Lys residue, or
b) R₅ does not relate to hydrocarbon chain of formula C₂H₂, or
c) at least one from R₆-R₁₀ does not relate to hydrogen.

3. Compound for use according to claim 1, wherein it is selected from:
tyrosyl-D-alanyl-glicyl-cinnamylpiperazine,
tyrosyl-D-threonyl-glicyl-phenylalanyl-cinnamylpiperazine,
tyrosyl-D-arginyl-cinnamylpiperazine, and tyrosyl-D-threonyl-cinnamylpiperazine,
L-tyrosyl-D-alanyl-L-phenylalanyl-cinnamylpiperazine,
L-tyrosyl-D-alanyl-L-tryptophyl-cinnamylpiperazine,
L-tyrosyl-D-threonyl-L-phenylalanyl-cinnamylpiperazine,
L-tyrosyl-D-threonyl-L-tryptophyl-cinnamylpiperazine,
L-tyrosyl-D-alanyl-glicyl-L-phenylalanyl-cinnamylpiperazine,
L-tyrosyl-D-alanyl-glicyl-L-tryptophyl-cinnamylpiperazine,
L-tyrosyl-D-threonyl-glicyl-L-phenylalanyl-cinnamylpiperazine,
L-tyrosyl-D-threonyl-glicyl-L-tryptophyl-cinnamylpiperazine.

4. Compound for use according to claim 1, wherein it is used in a form intended for oral, by injection, or intravenous administration.

5. Compound for use according to claim 1, wherein it is used in a form of oral tablet to be administered directly into gastrointestinal tract, as intravenous infusion for peripheral administration, or injection into the tumor and its vicinity.

6. Compound for use according to claim 1, wherein it is used in a form of multidrug pharmaceutical composition, preferably containing additionally an analgesic opioid, and/or an anticancer drug.

7. Compound for use according to claim 1, wherein it is intended for use in classical chemotherapy of intestinal cancer, applied prior to surgery (neo-adjuvant therapy), or post-surgery (adjuvant therapy), or in chemotherapy of inoperable intestinal cancer, or as its supporting therapy, or it is intended for intestine protection from metastasis of cancer of different tissue origin.

8. Compound for use according to claim 1, wherein it is intended for use in classical chemotherapy of pancreatic cancer, applied prior to surgery (neo-adjuvant therapy), or post-surgery (adjuvant therapy).

9. Compound of general formula: 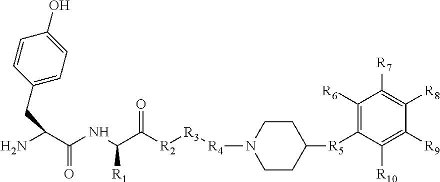
wherein:
R₁ relates to side chain of D-amino acid, selected from: D-Ala, D-Tre, D-Ser, D-Met, D-Leu, D-Glu, D-Asp, D-Lys or D-Arg,
R₂ relates to none, or to L-amino acid residue selected from Gly or Lys, or dipeptide residue L-Gly-L-Lys,
R₃ relates to L-amino acid residue selected from Phe or Trp,
R₄ relates to none, or to L-amino acid residue Lys,
R₅ relates to straight, saturated or unsaturated hydrocarbon chain of general formula CₙHₘ, wherein n is an integer from 1 to 4, and m is an even integer from 2 to 10,
R₆₋₁₀ relates independently to a substituent selected from: hydrogen, hydroxyl, methyl, formyl, carboxy, methoxycarbonyl, carbamoyl, cyano, amino, methylamino, dimethylamino, iodo, fluoro, nitro, or sulphone,
except of compound of general formula: 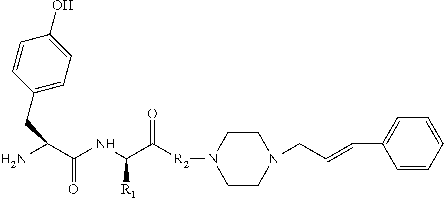
wherein:
R₁ relates to side chain of D-amino acid selected from: D-alanine, D-threonine, D-serine, D-methionine, D-leucine, D-glutamine, D-asparagine, D-lysine, or D-arginine,
and R₂ relates to none, or to residue of glycine, or a dipeptide selected from Gly-Phe or Gly-Trp.

10. Compound according to claim 9, wherein it is selected from:
L-tyrosyl-D-alanyl-phenylalanyl-cinnamylpiperazine,
L-tyrosyl-D-alanyl-L-tryptophyl-cinnamylpiperazine,
L-tyrosyl-D-threonyl-L-phenylalanyl-cinnamylpiperazine,
L-tyrosyl-D-threonyl-L-tryptophyl-cinnamylpiperazine.

11. Compound according to claims 9-10 for use as an antagonist of opioid drugs.

12. Compound for use according to claim 11, wherein, it is intended for oral or peripheral administration, to eliminate side effects of opioid drugs, especially intestinal disorder, constipation, or respiratory depression.

13. Compound for use according to claim 11, wherein, it is intended for direct or indirect interaction with opioid receptors.

14. Compound for use according to claim 11, wherein, it is used in a form of oral tablet to be administered directly into gastrointestinal tract, or as intravenous infusion for peripheral administration.

15. Compound for use according to claim 11, wherein, it is used in a form of multidrug pharmaceutical composition, containing, preferably, an opioid used in painkilling therapy.

16. Compound for use according to claim 1, or claim 11, wherein, it is used in a form of composition containing a polymer as a carrier for the active agent.
